(19) United States
(12) Patent Application Publication
(10) Pub. No.: US 2005/0025705 A1
(43) Pub. Date: Feb. 3, 2005

(54) Thrombosis animal models and their use in drug discovery and development

(76) Inventor: Xinkang Wang, Berwyn, PA (US)

Correspondence Address: STEPHEN B. DAVIS;BRISTOL-MYERS SQUIBB COMPANY, PATENT DEPARTMENT, P O BOX 4000, PRINCETON, NJ, 08543-4000, US

(21) Appl. No.: 10/896,383

(22) Filed: Jul. 22, 2004

Related U.S. Application Data
(60) Provisional application No. 60/489,311, filed on Jul. 22, 2003.

(51) Int. Cl. A61K 49/00; A61K 33/26

(52) U.S. Cl. 424/9.2; 424/647

(57) ABSTRACT

The present invention includes animal models useful for the study of thrombosis and the use of such models in drug discovery and development. The present invention relates to the induction of thrombus formation in a murine animal model by contacting a blood vessel with a low concentration of ferric chloride (FeCl₃). It is found for the first time in the present invention that the use of low ferric chloride concentrations permits thrombus formation which can be treated by known antithrombotic agents at desirable concentrations. Thus, the present invention permits the study of compounds for their possible use as antithrombotic agents under conditions which provide clinically meaningful results. Compounds discovered using the inventive animal models and methods of treatment using such compounds are also included. Gunmetal mice useful as animal thrombosis models are also included.

### FIELD OF THE INVENTION

The present invention is directed to animal models useful for the study of thrombosis and the use of such models in drug discovery and development. Particularly, the present invention relates to the induction of thrombus formation in an animal model, such as a mouse, by contacting a blood vessel thereof with a low concentration of ferric chloride (FeCl₃). It is found for the first time in the present invention that the use of low ferric chloride concentrations permits thrombus formation which can be treated by known antithrombotic agents at desirable concentrations. Thus, the present invention permits the study of compounds for their possible use as antithrombotic agents under conditions which provide clinically meaningful results.

### BACKGROUND OF RELATED TECHNOLOGY

Under normal circumstances, injury to vascular endothelial cells lining a blood vessel triggers a hemostatic response through a sequence of events commonly referred to as the coagulation “cascade”. This cascade culminates in the conversion of soluble fibrinogen to insoluble fibrin, which, together with platelets, form a localized clot or thrombus which prevents extravasation of blood components. Wound healing then occurs followed by clot dissolution and restoration of blood vessel integrity and flow.

Those events which occur between injury and clot formation are a carefully regulated and linked series of reactions. Briefly, a number of plasma coagulation proteins in inactive proenzyme form and cofactors circulate in the blood. Active enzyme complexes are assembled at the injury site and are sequentially activated to serine proteases, with each successive serine protease catalyzing the subsequent proenzyme to protease activation. This enzymatic cascade results in each step magnifying the effect of the succeeding step.

While efficient clotting limits the loss of blood at an injury site, inappropriate formation of thrombi in veins or arteries is a common cause of disability and death. Abnormal clotting activity can result in pathologies such as myocardial infarction, unstable angina, atrial fibrillation, stroke, renal damage, percutaneous translumenal coronary angioplasty, disseminated intravascular coagulation, sepsis, pulmonary embolism, atherosclerotic plaque rupture and deep vein thrombosis. Abnormal clotting can also result from treatments including the formation of clots on foreign surfaces of artificial organs, shunts and prostheses such as artificial heart valves.

Approved anticoagulant agents currently used in the treatment of these pathologies and other thrombotic and embolic disorders include the sulfated heteropolysaccharides heparin and low molecular weight (LMW) heparin. These agents are administered parenterally and can cause rapid and complete inhibition of clotting by activation of the thrombin inhibitor, antithrombin III, and inactivation of all of the clotting factors.

Due to their potency, however, heparin and LMW heparin have undesirable side effects. For example, uncontrolled bleeding is a major complication that may result from the simple stresses of motion and contact with physical objects at a surgical site. It is observed in 1-7% of patients receiving continuous infusion and in 8-14% of patients given intermittent bolus doses. To minimize this risk, samples are continuously drawn to enable ex vivo clotting times to be continuously monitored. However, this adds substantially to the cost of therapy and is inconvenient. Further, the therapeutic target range to achieve the desired level of efficacy without placing the patient at risk for bleeding is narrow. The therapeutic range is approximately 1 to less than 3 ug heparin/ml plasma which results in activated partial thromboplastin time (aPTT) assay times of about 35-100 seconds. Increasing the heparin concentration to 3 ug/ml exceeds the target range and at concentrations greater than 4 ug/ml, clotting activity is not detectable. Thus, great care must be taken to keep the patient's plasma concentrations within the therapeutic range.

Another approved anticoagulant with slower and longer lasting effects is Warfarin, a Coumadin® derivative. Warfarin acts by competing with Vitamin K-dependent post-translational modification of prothrombin and other Vitamin K-dependent clotting factors. The general pattern of anticoagulant action, in which blood is rendered non-clottable at concentrations only slightly higher than the therapeutic range, is seen for Warfarin as well as for heparin and LMW heparin.

In acute myocardial infarction (MI), the major objectives of thrombolytic therapy include early and sustained reperfusion of the infarcted vessel. Present therapy for acute MI includes both a plasminogen activator, such as tissue plasminogen activator (tPA) or streptokinase and an anticoagulant such as unfractionated heparin, low molecular weight heparin or direct thrombin inhibitors or antiplatelet agents such as aspirin or platelet glycoprotein IIb/IIIa blocker. (Topol, Am. Hear.t J., (1998) 136:S66-S68). This combination of therapies is based on the observation that clot formation and dissolution are dynamic processes and that thrombin activity and generation continue after the formation of the occlusive thrombus and during and after dissolution of the clot. (Granger et al., J. Am. Coll. Cardiol., (1998) 31:497-505).

The optimal strategy for treatment of acute MI remains elusive and available agents and treatment protocols display both negative and positive characteristics. For example, fibrin-bound thrombin is insensitive to inhibition by heparin (Becker et al., “Chemistry and Biology of Serpins”, (1997) Plenum Press, New York) and thrombin activity exhibits a rebound increase following cessation of heparin therapy with an observed increase in reinfarction within 24 hours following discontinuation of heparin. (Watkins et al., Catheterization and Cardiovascular Diagnosis, (1998) 44:257-264; Granger, Circulation, (1995) 91:1929-1935). Further, antiplatelet agents may be accompanied by bleeding or thrombocytopenia.

Also, numerous clinical trials have shown that high doses of thrombolytic agents lead to significant alteration in plasma hemostatic markers. (Rao et al., J. Clin. Invest., (1988) 101:10-14; Bovill et al., Ann. Int. Med., (1991) 115:256-265; Neuhaus et al., J. Am. Coll. Cardiol., (1992) 19:885-891). Although increasing concentrations of tPA leads to enhanced clot dissolution, the alteration in these hemostatic markers mirrors increased liabilities of thrombolytic therapy, particularly the incidence of severe bleeding.

Animal models have played a crucial role in drug discovery and development. Many antithrombotic agents have been initially validated in various animal models of thrombosis and successfully launched for the treatment and/or prevention of thrombotic diseases in clinic (Leadley et al., J. Pharmacol. Toxicol. Methods, (2000) 43:101-116), such as Activase® (recombinant tissue plasminogen activator; Matsuo et al., Nature, (1981) 291:590-591), Abciximab (Coller et al., Blood, (1986) 68:783-786) and Hirudin (Agnelli et al., Thromb. Haemost., (1990) 63:204-207).

Additionally, animal models are of great value in target validation due to recent advances in genetics and molecular biology that permit the development of transgenic animals with knock-out or over-expression of particular genes, such as those related to thrombosis and hemostasis. (Leadley et al., (2000) J. Pharmacol. Toxicol. Methods, 43:101-116; Hogan et al., Thromb. Haemost., (2002) 87:563-574). The use of animal models of thrombosis is one of the first key steps in validating novel therapeutic targets in drug discovery and development. Animal models of thrombosis have been developed and successfully used to evaluate therapeutic drugs in various species, including rat and rabbit. (Leadley et al., J. Pharmacol. Toxicol. Methods, (2000) 43:101-116). However, there is presently no effective mouse model of thrombosis.

For example, ferrous chloride-induced (Heran et al., Eur. J. Pharmacol., (2000) 389:201-207) and electrolytic injury-induced (Kawasaki et al., Throm. Haemost., (1998) 79:410-416) carotid artery thrombosis models have been used to evaluate factor Xa inhibitors in rodents (rats). A rabbit model of arterial-venous shunt thrombosis has also been developed. (Wong et al., J. Pharmacol. Experimental Therapeutics, (2000) 292:351-357).

However, the current rodent (murine) models of thrombosis are not sensitive enough to be predictive of clinical results. While ferric chloride-induced arterial thrombosis has been reported (Zhu et al., Circulation, (1999) 99:3050-3055; Konstantinides et al., Circulation, (2001) 103:576-583), such reports utilized high concentrations (10-20%) of a ferric chloride solution, resulting in the formation of a thrombus that is insurmountable for many compounds of interest, causing misleading results for investigators as to the efficacy of compounds of interest.

Moreover, platelets play a critical role in hemostasis and thrombosis. The activation of platelets is one of the key components resulting in myocardial infarction, unstable angina, deep vein thrombosis, and stroke. Mice homozygous for a spontaneous and recessive mutation gunmetal (gm) have been identified to have prolonged bleeding due to defects in platelets and megakaryocytes (Swank R. T., et al., Blood May 15, 1993;81(10):2626-35; Novak E. K., et al., Blood Apr 1, 1995;85(7):1781-9). Gunmetal mice also showed macrothrombocytopenia and reduced platelet α- and δ-granule contents, characterized as the storage pool diseases (SPDs) in patients (Reed G. L., et al., Blood 2000;96:3334-42).

Gunmetal resulted from a G to A substitution mutation in a splice receptor site within the α-subunit of Rab geranylgeranyl transferase (Rabggtase), an enzyme that attaches geranylgeranyl groups to Rab protein (Detter J. C., et al., Proc. NatI. Acad. Sci. USA. 2000;97:4144-9). Gunmetal mice have reduced Rabggtase protein and enzyme activity in gunmetal platelets (Detter et al., 2000). The cell-specific abnormal prenylation of Rab protein in platelets and melanocytes of the gunmetal mice (Zhang Q., et al., Br. J. Haematol. 2002;117:414-23) suggests a critical role of Rabggtase in thrombocytosis and thrombosis. However, no direct evidence is available for a role of gunmetal in thrombosis.

Accordingly, the need exists to discover and develop antithrombotic agents that are efficacious in controlling thrombotic disorders while maintaining hemostatic functions. As present animal models have not proven entirely suitable for the discovery and development of such agents, there is a need to establish a simple yet sensitive murine animal model to determine the efficacy of compounds of interest, as well as to establish proof of concept studies using genetically manipulated mice.

### SUMMARY OF THE INVENTION

The present invention is directed to animal models useful for the study of thrombosis and the use of such models in drug discovery and development. Particularly, the present invention relates to the induction of thrombus formation in an animal model, such as a mouse, by contacting a blood vessel thereof with a low concentration (2-10%) of ferric chloride. It is found for the first time in the present invention that the use of low ferric chloride concentrations permits thrombus formation which can be treated by known antithrombotic agents at desirable concentrations. Thus, the present invention permits the study of compounds for their possible use as antithrombotic agents under conditions which provide clinically meaningful results.

In one aspect, the present invention is directed to a method of identifying a compound useful for treating or preventing an occlusion in a blood vessel of an animal, comprising: (a) exposing a blood vessel of an animal to a ferric chloride solution which comprises a concentration of from about 2% to about 10% ferric chloride, thereby inducing an occlusion in the blood vessel; (b) introducing a compound of interest to the animal; and (c) determining if the compound of interest affects a physical or chemical alteration of the occlusion.

In a desired aspect, the occlusion may be a thrombus and the animal may be non-human, such as a rodent, and more specifically may be a mouse. The blood vessel may be an arterial vessel, such as a carotid artery. The ferric chloride solution desirably comprises a concentration of from about 2.5% to about 7% ferric chloride and from about 2.5% to about 5% ferric chloride. The step of determining if the compound of interest affects a physical or chemical alteration in the occlusion may be determined by measuring a change in blood flow through the blood vessel, such as by using a Doppler flow probe and by measuring a change in weight of the thrombus.

In another aspect, the present invention is directed to a compound identified using the above method. The compound is desirably selected from the group consisting of anti-platelets, anticoagulants and prothrobolytics.

In another aspect, the present invention is directed to a method of treating or preventing thrombosis in an animal, comprising administering to the animal a compound above.

In another aspect, the present invention is directed to a method of identifying a compound useful for treating or preventing thrombosis in a mouse, comprising: (a) exposing an arterial vessel in a mouse to a ferric chloride solution comprising a concentration of from about 2% to about 5% ferric chloride, thereby inducing the formation of a thrombus in the arterial vessel; (b) introducing a compound of interest to the mouse; and (c) determining if the compound of interest affects a physical or chemical alteration of the thrombus.

In another aspect, the present invention is directed to an animal model useful for identifying a compound for treating or preventing an occlusion in a blood vessel of an animal, the animal model comprising an occlusion in a blood vessel which is formed by exposing the blood vessel to a ferric chloride solution comprising a concentration of from about 2% to about 10% ferric chloride.

In another aspect, the present invention is directed to an animal model useful for identifying a compound for treating or preventing an occlusion in a blood vessel of an animal, the animal model comprising: (a) exposing a blood vessel of an animal to a ferric chloride solution comprising a concentration of from about 2% to about 10% ferric chloride, thereby inducing an occlusion in the blood vessel; (b) introducing a compound of interest to the animal; and (c) determining if the compound of interest affects a physical or chemical alteration of the occlusion.

In another aspect, the present invention is directed to a method of identifying a compound useful for treating or preventing an occlusion in a blood vessel of an animal, the method comprising: (a) exposing a blood vessel of a gunmetal mouse to a ferric chloride solution, the ferric chloride solution comprising a concentration of from about 2% to about 10% ferric chloride, thereby inducing an occlusion in the blood vessel; (b) introducing a compound of interest to the animal; and (c) determining if the compound of interest affects a physical or chemical alteration of the occlusion.

### BRIEF DESCRIPTION OF THE DRAWINGS

FIG. 1 shows the concentration-dependent effects of FeCl₃ (2-10%) on arterial thrombosis in C57BL/6 mice.

FIG. 2 shows the effects of heparin on 2.5% and 5% FeCl₃-induced arterial thrombosis in mice.

FIG. 3 shows the effects of clopidogrel on 2.5% and 5% FeCl₃-induced arterial thrombosis in mice.

FIG. 4 shows the effects of aspirin and the combination of clopidogrel and aspirin on 2.5% FeCl₃-induced thrombosis in mice.

FIG. 5 shows the effects of cangrelor on 2.5% and 5% FeCl₃-induced arterial thrombosis in mice.

FIG. 6 shows the effects of MRS2179 on 2.5% FeCl₃-induced arterial thrombosis in mice.

FIG. 7 shows the effects of various antithrombotic agents on tail bleeding time in C57BL/6 mice.

FIGS. 8A and 8B show ferric chloride-induced arterial thrombosis in gunmetal mice and control littermates.

FIGS. 9A and 9B show ferric chloride-induced arterial thrombosis in C57BL/6 mice following clopidogrel treatment.

FIG. 10 shows tail bleeding time in gunmetal mice and C57BL/6 mice following clopidogrel treatment.

### DETAILED DESCRIPTION OF THE INVENTION

The present invention is directed to animal models useful for the study of thrombosis and the use of such models in drug discovery and development. Particularly, the present invention relates to the induction of thrombus formation in an animal model, such as a mouse, by contacting a blood vessel thereof with a low concentration (2-10%) of ferric chloride. It is found for the first time in the present invention that the use of low ferric chloride concentrations permits thrombus formation which can be treated by known antithrombotic agents at desirable concentrations. Thus, the present invention permits the study of compounds for their possible use as antithrombotic agents under conditions which provide clinically meaningful results.

It is presently accepted that animal models for discovery of antithrombotic agents include the use of a ferric chloride solution of 10% or greater ferric chloride to induce thrombus formation. It has been surprisingly found in the present invention, however, that this accepted standard is not sensitive enough to accurately identify possible antithrombotic agents in a clinically meaningful manner. Accordingly, the present invention is directed to a more sensitive model for use in thrombosis studies, particularly a mouse model which utilizes a ferric chloride concentration of about 2-10% ferric chloride. The use of such a concentration has been surprisingly found to permit the study of agents which are useful for thrombosis dissolution at concentrations which may also be suitable clinically, i.e., at concentrations which would not result in undesirable side effects. Thus, the present invention provides a more predictive model of thrombosis, as evidenced in the Examples set forth herein. The present Examples are meant to more fully illustrate desirable embodiments of the present invention are in no way meant to limit the scope of the present invention.

The following sets for the Materials and Methods used in the present invention, and which were utilized in the Examples set forth below.

Materials and Methods

1. Mice

C57BL/6 mice (18-22 g; Charles River Laboratories, Wilmington, Mass.) were used throughout the experiments. Animals were housed and cared for in accordance with the Guide for the Care and Use of Laboratory Animals [DHEW (DHHS) Publication No. (NIH) 85-23, revised 1996, Office of Science and Health Reports, DRR/NIH, Bethesda, Md. 20205]. Procedures using lab animals were approved by the Institutional Animal Care and Use Committee of Bristol-Myers Squibb Company.

Mice were anesthetized with gas inhalation comprised of 30% oxygen (0.3 liter/min; Airgas East, Inc., Salem, N.H.) and 70% nitrous oxide (0.7 liter/min; Airgas East, Inc., Salem, N.H.). The gas was passed through an isoflurane vaporizer (VetEquip Inc., Pleasanton, Calif.) set to deliver 3-4% isoflurane (isoflurane (Hanna's Pharm Supply Co., Wilmington, Del.) during initial induction and 1.5-2% during surgery. After anesthesia, an incision of the skin was made directly on top of the right common carotid artery region. The fascia was then blunt dissected and a segment of the left common carotid artery was exposed. Carotid blood flow was measured with a miniature Doppler flow probe (Model 0.5 VB, Transonic System Inc., Ithaca, N.Y.) as described previously (Zhu et al., Circulation (1999) 99:3050-3055). Thrombosis was induced by applying a filter paper (0.5×1 mm) saturated with various concentrations (2, 2.4, 2.5, 3, 5 and 10%) of ferric chloride (Sigma Co., St. Louis, Mo.) to the adventitial surface of the carotid artery immediately proximal to the flow probe for 3 min and then removed. The carotid blood flow was monitored at 1, 5, 10, 20, 30 min or some times up to 60 min.

2. Drug Administration

Heparin was purchased from American Pharmaceutical Partners, Inc. (Los Angeles, Calif.); mice were infused with 10, 50, 200 and 1000 IU/kg, i.v. immediately prior to 2.5 or 5% FeCl₃ treatment on the carotid artery. Clopidogrel (Plavix®) was produced by Sanofi Pharmaceuticals, Inc. (New York, N.Y.); the drug was pulverized using a porcelain mortar and pestle and the powder was re-suspended in saline and administered into mice for two days at 1, 3, 10, 30 and 100 mg/kg, p.o., of which the second dose was given at 2-4 hours prior to the induction of thrombosis. Aspirin (acetylsalicylic acid) and MRS2179 were purchased from Sigma. Aspirin was administrated into mice at 10 and 30 mg/kg, i.p. 0.2-2 hours prior to FeCl₃ treatment, whereas MRS2179 was injected into the jugular vein 30 seconds before thrombosis induction.

3. Statistical Analysis

Data in text and figures are mean±standard errors for the indicated number (N) of animals. Statistical comparisons were made by analysis of variance (ANOVA; Fisher's protected least squares difference) and values were considered to be significant at p<0.05.

### EXAMPLE 1

### Concentration-Dependent Effect of FeCl₃ on Arterials Thrombosis in Mice

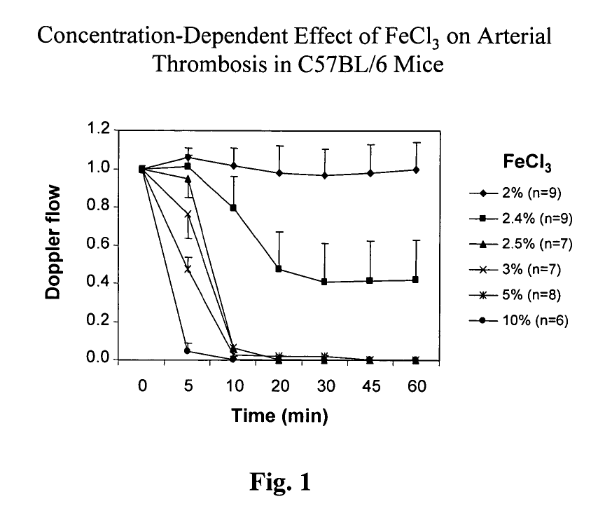

Concentration-dependent effects of FeCl₃ (2-10%) on thrombus formation, as reflected by the blood flow measurement, in the carotid artery were demonstrated in C57BL/6 mice, as shown in FIG. 1. No reduction in blood flow was observed following 2% FeCl₃ treatment. At 2.4% FeCl₃ concentration, 2 out of 9 animals were completely occluded at 10 min and 6 animals occluded at 30 min, while 3 animals remained vascular patency throughout the time course (up to 60 min). A threshold stimulus was reached at 2.5% FeCl₃, showing a marked reduction in blood flow 10 min. post treatment in every animal tested. A robust difference in blood flow was observed at 5 min following the treatment of various FeCl₃ concentrations (2.5-10%), while almost all the vessels were completely occluded at 10 min.

### EXAMPLE 2

### Effect of Various Agents on FeCl₃-Induced Arterial Thrombosis in Mice

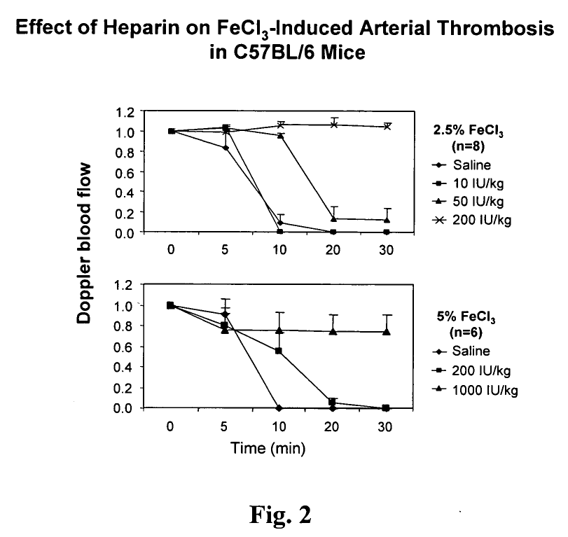

To demonstrate the utility of the inventive arterial thrombosis models for antithrombotic drug assessment, various agents, including heparin (an anticoagulation agent), clopidogrel (a selective P2Y12 antagonist), aspirin (a cyclooxygenase/TXA2 inhibitor), cangrelor (a selective P2Y12 receptor antagonist) and MRS2179 (a selective P2Y1 antagonist) were used in 2.5 and 5% FeCl₃-induced arterial thrombosis. FIG. 2 shows the effect of heparin on FeCl₃-induced arterial thrombosis.

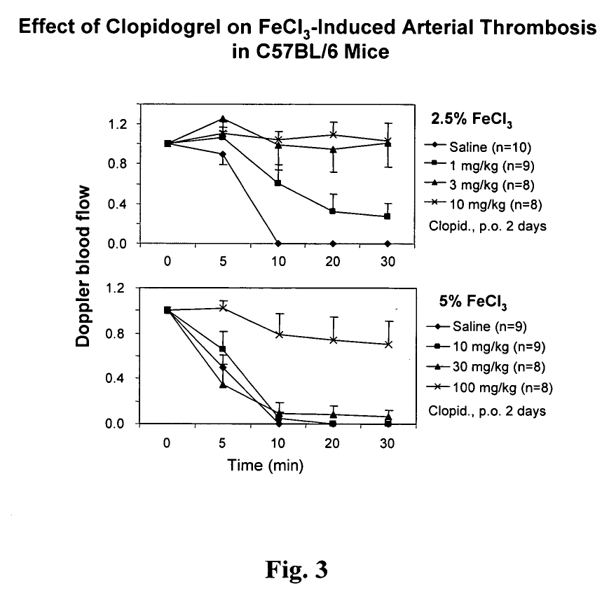

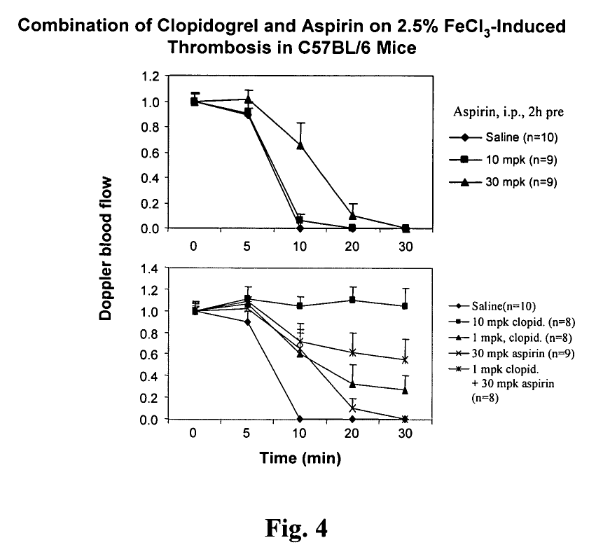

Vascular patency was maintained in mice treated with 200 IU/kg heparin, i.v. in the 2.5% FeCl₃-induced thrombosis; while an extremely high concentration of heparin (1000 IU/kg) was required to inhibit 5% FeCl₃-induced thrombosis (FIG. 2). Similarly, vascular patency was maintained in some of 1 mg/kg clopidogrel (an selective P2Y receptor antagonist), p.o. treated animals and all of 3 mg/kg clopidogrel treated animals in the model of 2.5% FeCl₃-induced thrombosis; but 100 mg/kg clopidogrel, p.o. was required to show efficacy in 5% FeCl₃-induced thrombosis (FIG. 3). Very little effect was observed for aspirin treatment in blocking 2.5% FeCl₃-induced thrombosis (FIG. 4, upper panel). The combination of aspirin (30 mg/kg, i.p.) and clopidogrel (1 mg/kg, p.o.) revealed an additive effect in blocking thrombosis (FIG. 4, lower panel). However, no efficacy was observed at 10% FeCl₃-induced thrombosis even if the mice were treated with the combination of 100 mg/kg clopidogrel and 30 mg/kg aspirin (data not shown).

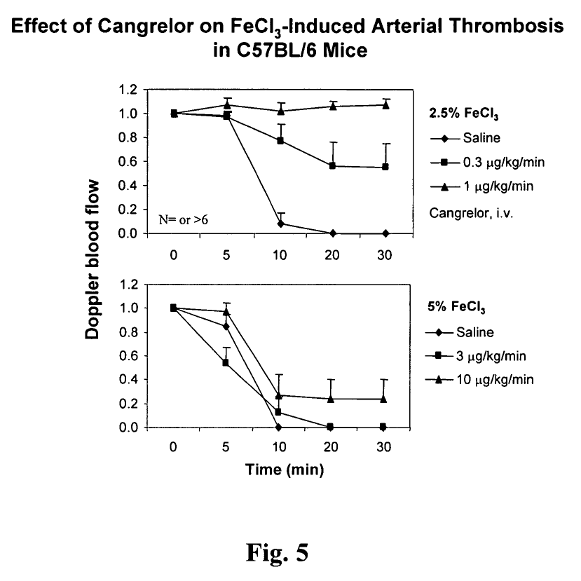

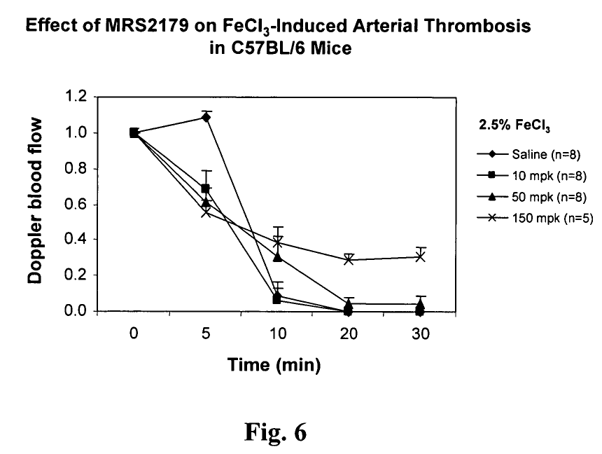

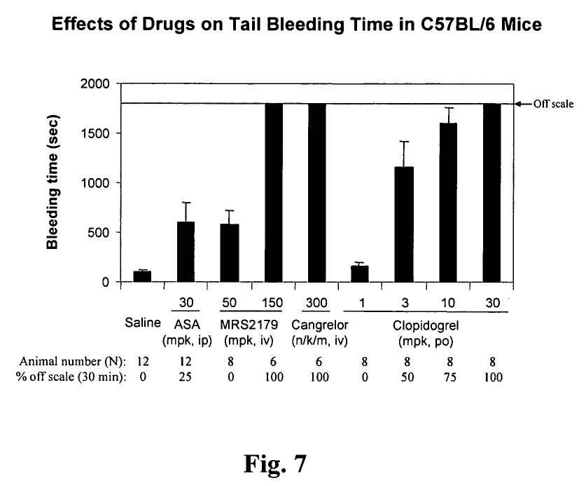

Also shown is the differential efficacy of cangrelor, another selective P2Y12 antagonist, in 2.5% and 5% FeCl₃-induced thrombosis (FIG. 5). In contrast, P2Y1 antagonist, MRS2179 , appeared to be less potent in blocking FeCl₃-induced thrombosis (FIG. 6), since the effective dose of MRS2179 (i.e., 150 mg/kg, i.v.) might result in bleeding liability (FIG. 7).

### EXAMPLE 3

### Gunmetal Mouse Model

Gunmetal (gm) mice exhibit reduced rates of platelet synthesis and decreased platelet α- and δ-granule contents. Its genotype has been associated with a mutation in the Rab geranylgeranyl transferase (Rabggtase) gene that encodes an enzyme attaches geranylgeranyl groups to Rab proteins. Evaluation of the effect of gunmetal on thrombosis using a murine model of ferric chloride-induced carotid artery thrombosis was conducted.

Significant protection was observed in gm/gm mice in 5% ferric chloride-induced arterial thrombosis compared to its +/gm and +/+littermates. The level of this protection in gunmetal mice was similar to that following the treatment of a high dose of P2Y12 antagonist clopidogrel (30-100 mg/kg) in C57BL/6 mice. Tail transaction studies showed a dose-dependent effect of Rabggtase gene in bleeding time, with 4- and 12-fold increase in +/gm and gm/gm mice over wild-type littermates, respectively.

C57BL/6J mice herterozygous for the congenic pigment mutation gunmetal were originally obtained from Jackson Laboratories (Bar Harbor, Me.) and were back-crossed to at least 10th generation against C57BL/6 mice in the animal facility of Harvard School of Public Health. Male and female gm/gm, +/gm and +/+littermates were used for the study at 6-12 weeks of age. Animals were housed in microisolation cages on a constant 12-hour light/dark cycle with controlled temperature and humidity and given access to food and water ad libitum. C57BL/6 mice were obtained from Jackson Laboratories and used to establish the thrombosis model and to assess the effect of clopidogrel.

Animals were housed and cared for in accordance with the Guide for the Care and Use of Laboratory Animals [DHEW (DHHS) Publication No. (NIH) 85-23, revised 1996, Office of Science and Health Reports, DRR/NIH, Bethesda, Md. 20205]. Procedures using lab animals were approved by the Institutional Animal Care and Use Committee of Bristol-Myers Squibb Company and Harvard School of Public Health.

a. Thrombosis Model

Adult mice (18-25 g, both male and female) were used for thrombosis study. Following anesthesia by pentobarbital (50 mg/kg, i.p.), an incision of the skin was made directly on top of the right common carotid artery region. The fascia was then blunt dissected and a segment of the left common carotid artery was exposed. A Transonic flow probe (0.5 VB) was placed on the artery with gel to obtain the baseline flow readings (as time 0) via a Transonic T106 Doppler flowmeter. Two pieces of filter paper (1×1½ mm) pre-soaked with 5% FeCl₃ solution were placed beneath and above the carotid artery for 3 min to induce thrombosis. The filter papers were removed and rinsed with saline. The blood flow was recorded every 5, 10, 20 and 30 min of FeCl₃ treatment.

b. Mouse Tail Bleeding

Mice were anesthetized as described above and placed on a 37° C. heating pad. About 2-4 mm from the tip of mouse tail (in about 1 mm diameter) was cut with a disposable surgical blade. After transection, the tail was immediately placed into a 50-ml falcon tube filled with 37° C. saline and the bleeding time was recorded up to 30 min. Bleeding time over 30 min was considered as bleeding off scale.

c. Drug Administration

Clopidogrel (Plavix®) was purchased from Sanofi Pharmaceuticals, Inc (New York, N.Y., USA) and the tablets were pulverized using a porcelain and mortar and dissolved in water in appropriate concentration for p.o. dosing in a volume of 10 ml/kg. Clopidogrel was administrated for two days. Both thrombosis and bleeding studies were conducted within 2-4 hr after the second day of oral dosing.

d. Statistical Analysis

Data are illustrated as mean±standard errors using indicated number (N) of animals. Statistical comparisons were made by analysis of variance (ANOVA; Fisher's protected least squares difference) and values were considered to be significant at p<0.05.

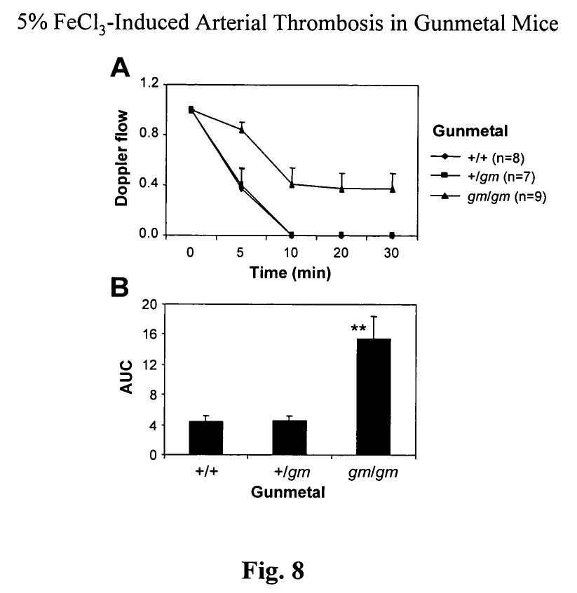

As illustrated in FIG. 8, 5% FeCl₃ was used to induce arterial thrombosis in gunmetal and C57BL/6 mice. The carotid artery was dissected and subjected to thrombosis induced with 5% FeCl₃ in gunmetal homozygous (gm/gm), heterozygous (+/gm) and wild-type (+/+) mice (A and B). Doppler blood flow was measured prior to (as the base line, time 0), and at 5, 10, 20 and 30 min after 3 min-FeCl₃ treatment. Vessel patency was retained in 5 out of 9 gunmetal mice. Data are illustrated as the relative Doppler blood flow (A) or the area under curve (AUC) (B). The mean values +standard errors of each experimental group (the number of animals are indicated) are illustrated. Complete vessel occlusion occurred within 10 min following FeCl₃ treatment in both wild-type (n=8) and heterozygous (n=7) mice, while the vascular patency was retained in 4 out of 9 gunmetal mice over the time course observed (FIG. 8A). The area under curve (AUC) of the relative Doppler blood flow showed a significant protection of gunmetal mice from the arterial thrombosis (3.5-fold over wild-type animals, p<0.01; FIG. 8B). No protection was observed in the heterozygous mice.

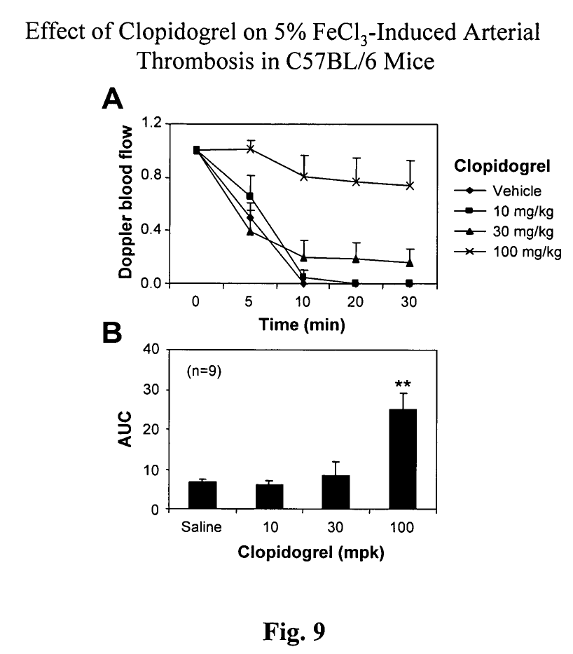

Under a similar experimental condition, a P2Y12 receptor antagonist, clopidogrel, was used as an antiplatelet agent to assess its effect on 5% FeCl₃-induced arterial thrombosis in C57BL/6 mice. No antithrombotic effect was observed for clopidogrel in the this model at a clinically relevant dose (1 mg/kg), unless a very high concentration (>30 mg/kg, p.o.) was used. As noted, 2 of out 9 animals in the 30 mg/kg clopidogrel group or 6 out of 9 animals in the 100 mg/kg clopidogrel group retained vascular patency (FIG. 9A). Significant protection against thrombosis was observed only in the 100 mg/kg clopidogrel group examined by the AUC of the relative Doppler blood flow (3.7-fold over controls, p<0.01; FIG. 9B).

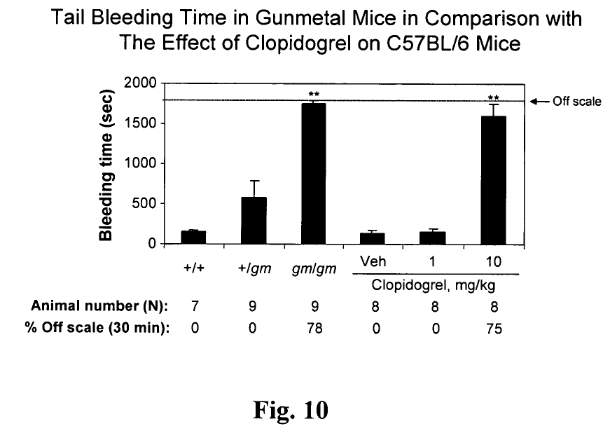

Tail bleeding study was used to assess the disturbance of hemostasis in gunmetal mice and for the effect of clopidogrel in C57BL/6 mice. Tails of gunmetal homozygous (gm/gm), heterozygous (+/gm) and wild-type littermates (+/+) or C57BL/6 mice following vehicle (water), 1 and 10 mg/kg clopidogrel, p.o. treatments were cut and bleeding time was measured. The number of animals in each group was indicated. The maximal bleeding time was set at 30 min, and the percentage of animal reached the maximal bleeding time (off scale) in each group is indicated. Marked prolongation in bleeding time was observed in heterozygous (4-fold increase over wild-type animals, p=0.07) and even more profound effect in gunmetal mice (12-fold increase with 78% animals bleeding off scale, p<0.01, n=9). While no prolongation in bleeding time was observed following 1 mg/kg clopidogrel treatment, exacerbated bleeding was observed following 10 mg/kg clopidogrel treatment (11-fold increase and 75% animals bleeding off scale, p<0.01, n=8) (FIG. 10). The dose of 30 mg/kg clopidogrel resulted in maximal bleeding time, i.e., 100% animals bleeding off scale (data not shown).

### Discussion

In the present invention, the creation of novel models of arterial thrombosis in mice by using various concentration of FeCl₃ in the carotid artery has been described. A threshold of FeCl₃ concentration was identified to induce vascular thrombosis and the concentration slightly above this threshold point, i.e., 2.5% FeCl₃, was demonstrated to be consistent and very sensitive to various antithrombotic agents and therefore is useful for antithrombotic drug discovery.

Further, prior to the present invention there has been no direct evidence for a role of gunmetal in thrombosis. The present invention shows significant protection of gunmetal mice from FeCl₃-induced arterial thrombosis. The degree of this protection resembles a high dose of clopidogrel (30-100 mg/kg) in the same animal model in C57BL/6 mice. This antithrombotic effect, however, was not observed in heterozygous or wild-type littermates of gunmetal mice.

Previous report showed that the gunmetal mutation reduced cellular levels of the a subunit of Rabggtase to about 20% in gm/gm and 60% in +/gm of normal (Detter et al., 2000). The lose of antithrombotic efficacy in heterozygous gunmetal mice might be explained by the severe condition (5% FeCl₃) used to induce the arterial thrombosis. As demonstrated in this work, the antithrombotic efficacy was not observed for clopidogrel in 5% FeCl₃-induced thrombosis unless a much higher than therapeutic dose was used.

In contrast, the dose-dependent effect of gunmetal was obvious on hemostasis, where +/gm and gm/gm gunmetal mice showed 4- and 12-fold increase in bleeding time over controls, respectively. This dose-dependent effect on bleeding time was in accordance with its effect Rabggtase (Detter et al., 2000). While 10 mg/kg clopidogrel failed to protect from 5% FeCl₃-induced thrombosis, it significantly increased (10.9-fold) in bleeding time in C57BL/6 mice, suggesting a potentially better margin of efficacy/safety for Rabggtase blockade than P2Y1 as therapeutic agents.

Accordingly, as demonstrated in the present invention, when relatively high FeCl₃ concentrations were used to induce arterial thrombosis most antithrombotic agents (such as heparin, clopidogrel, aspirin, cangrelor and MRS2179) failed to show efficacy in blocking thrombus formation, unless an extremely high drug concentration was used. The use of a high drug concentration is unacceptable, however, due to bleeding liability. The concentration-dependent effect of FeCl₃ on thrombosis induction and its differential effects by various antithrombotic agents in mice has not been previously known. For the first time, the present invention establishes that an appropriate concentration of FeCl₃, such as 2.5-5%, for the induction of arterial thrombosis may be adapted for antithrombotic drug discovery and validation.

While the invention has been described in connection with specific embodiments therefore, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims. All references cited herein are expressly incorporated in their entirety.

1. A method of identifying a compound useful for treating or preventing an occlusion in a blood vessel of an animal, comprising:
a. exposing a blood vessel of an animal to a ferric chloride solution, said ferric chloride solution comprising a concentration of from about 2% to about 10% ferric chloride, thereby inducing an occlusion in said blood vessel;
b. introducing a compound of interest to said animal;
c. determining if said compound of interest affects a physical or chemical alteration of said occlusion.

2. The method of claim 1, wherein said occlusion is a thrombus.

3. The method of claim 1, wherein said animal is non-human.

4. The method of claim 1, wherein said animal is a rodent.

5. The method of claim 4, wherein said rodent is a mouse.

6. The method of claim 1, wherein said blood vessel is an arterial vessel.

7. The method of claim 6, wherein said arterial vessel is a carotid artery.

8. The method of claim 1, wherein said ferric chloride solution comprises a concentration of from about 2.5% to about 7% ferric chloride.

9. The method of claim 1, wherein said ferric chloride solution comprises a concentration of from about 2.5% to about 5% ferric chloride.

10. The method of claim 1, wherein said step of determining if said compound of interest affects a physical or chemical alteration in said occlusion is determined by measuring a change in blood flow through said blood vessel.

11. The method of claim 10, wherein said measuring a change in blood flow through said blood vessel is performed using a Doppler flow probe.

12. The method of claim 1, wherein said step of determining if said compound of interest affects a physical or chemical alteration of said occlusion is determined by measuring a change in weight of said thrombus.

13. A compound identified using the method of claim 1.

14. The compound of claim 13, wherein said compound is selected from the group consisting of anti-platelets, anticoagulants and prothrobolytics.

15. A method of treating or preventing thrombosis in an animal, comprising administering to said animal a compound of claim 13.

16. A method of identifying a compound useful for treating or preventing thrombosis in a mouse, comprising:
a. exposing an arterial vessel in a mouse to a ferric chloride solution, said ferric chloride solution comprising a concentration of from about 2% to about 5% ferric chloride, thereby inducing the formation of a thrombus in said arterial vessel;
b. introducing a compound of interest to said mouse;
c. determining if said compound of interest affects a physical or chemical alteration of said thrombus.

17. An animal model useful for identifying a compound for treating or preventing an occlusion in a blood vessel of an animal, said animal model comprising an occlusion in a blood vessel, wherein said occlusion is formed by exposing said blood vessel to a ferric chloride solution comprising a concentration of from about 2% to about 10% ferric chloride.

18. The animal model of claim 17, wherein said occlusion is a thrombus.

19. The animal model of claim 17, wherein said animal is non-human.

20. The animal model of claim 17, wherein said animal is a rodent.

21. The animal model of claim 20, wherein said rodent is a mouse.

22. The animal model of claim 17, wherein said blood vessel is an arterial vessel.

23. The animal model of claim 22, wherein said arterial vessel is a carotid artery.

24. The animal model of claim 17, wherein said ferric chloride solution comprises a concentration of from about 2.5% to about 7% ferric chloride.

25. The animal model of claim 17, wherein said ferric chloride solution comprises a concentration of from about 2.5% to about 5% ferric chloride.

26. A compound identified using the animal model of claim 17.

27. The compound of claim 26, wherein said compound is selected from the group consisting of anti-platelets, anticoagulants and prothrobolytics.

28. A method of treating or preventing thrombosis in an animal, comprising administering to said animal a compound of claim 26.

29. An animal model useful for identifying a compound for treating or preventing an occlusion in a blood vessel of an animal, said animal model comprising:
a. exposing a blood vessel of an animal to a ferric chloride solution, said ferric chloride solution comprising a concentration of from about 2% to about 10% ferric chloride, thereby inducing an occlusion in said blood vessel;
b. introducing a compound of interest to said animal;
c. determining if said compound of interest affects a physical or chemical alteration of said occlusion.

30. The animal model of claim 29, wherein said occlusion is a thrombus.

31. The animal model of claim 29, wherein said animal is non-human.

32. The animal model of claim 31, wherein said animal is a rodent.

33. The animal model of claim 32, wherein said rodent is a mouse.

34. The animal model of claim 29, wherein said blood vessel is an arterial vessel.

35. The animal model of claim 34, wherein said arterial vessel is a carotid artery.

36. The animal model of claim 29, wherein said ferric chloride solution comprises a concentration of from about 2.5% to about 7% ferric chloride.

37. The animal model of claim 29, wherein said ferric chloride solution comprises a concentration of from about 2.5% to about 5% ferric chloride.

38. A compound identified using the animal model of claim 29.

39. The compound of claim 38, wherein said compound is selected from the group consisting of anti-platelets, anticoagulants and prothrobolytics.

40. A method of treating or preventing thrombosis in an animal, comprising administering to said animal a compound of claim 38.

41. A method of identifying a compound useful for treating or preventing an occlusion in a blood vessel of an animal, comprising:
a. exposing a blood vessel of a gunmetal mouse to a ferric chloride solution, said ferric chloride solution comprising a concentration of from about 2% to about 10% ferric chloride, thereby inducing an occlusion in said blood vessel;
b. introducing a compound of interest to said animal;
c. determining if said compound of interest affects a physical or chemical alteration of said occlusion.

42. The method of claim 41, wherein said ferric chloride solution comprises a concentration of from about 2.5% to about 7% ferric chloride.

43. The method of claim 41, wherein said ferric chloride solution comprises a concentration of from about 2.5% to about 5% ferric chloride.

44. The method of claim 41, wherein said step of determining if said compound of interest affects a physical or chemical alteration in said occlusion is determined by measuring a change in blood flow through said blood vessel.

45. The method of claim 41, wherein said step of determining if said compound of interest affects a physical or chemical alteration of said occlusion is determined by measuring a change in weight of said thrombus.

46. A compound identified using the method of claim 41.

47. The compound of claim 46, wherein said compound is selected from the group consisting of anti-platelets, anticoagulants and prothrobolytics.

48. A method of treating or preventing thrombosis in an animal, comprising administering to said animal a compound of claim 46.
